(19) United States
(12) Patent Application Publication
(10) Pub. No.: US 2026/0146272 A1
(43) Pub. Date: May 28, 2026

(54) ENZYMATIC SYNTHESIS OF MODIFIED NUCLEOSIDE TRIPHOSPHATE ANALOGUES

(71) Applicant: Illumina, Inc., San Diego, CA (US)

(72) Inventors: Sadeer Salam, Singapore (SG); Xiangyuan Yang, Singapore (SG); Yvonne Devadas, Singapore (SG); Yin Nah Teo, Singapore (SG); Saurabh Nirantar, Singapore (SG); Ramesh Neelakandan, Singapore (SG); Hassan Bohra, Singapore (SG)

(21) Appl. No.: 19/394,058

(22) Filed: Nov. 19, 2025

Related U.S. Application Data
(60) Provisional application No. 63/724,222, filed on Nov. 22, 2024.

(51) Int. Cl. C12P 19/38; C12N 9/12

(52) U.S. Cl. CPC C12P 19/385; C12N 9/1223; C12N 9/1229

(57) ABSTRACT

The present disclosure generally relates to methods for the enzymatic synthesis of nucleotide triphosphates. In particular, the methods include a one-pot preparation of a modified nucleotide triphosphate starting from 3′-vinyl blocked nucleoside monophosphate.

### INCORPORATION BY REFERENCE TO ANY PRIORITY APPLICATION

The present application claims the benefit of priority to U.S. Ser. No. 63/724,222, filed Nov. 22, 2024, which is incorporated by reference in its entirety.

### BACKGROUND

### Field

The present disclosure generally relates to methods for the preparation of nucleoside triphosphates. In particular, the present disclosure is directed to the preparation of modified nucleoside triphosphates from modified nucleoside monophosphates via enzymatic synthesis.

### Description of the Related Art

Nucleoside triphosphates (NTP) and deoxynucleoside triphosphates (dNTP) are important building blocks of biomolecules, playing critical roles in cellular functions, and their use has been applied towards a wide field of scientific research and technology. Nucleoside-5′-triphosphates and their analogues are precursors in DNA synthesis and are key reagents for molecular biological research.

NTPs are primarily synthesized by chemical means. Generally, one popular method can be described by reacting phosphorus oxychloride (POCl₃) with the nucleoside dissolved in organic solvents; prepared in a moisture-free environment. Yoshikawa et al., Tet. Lett. 1967 8(50): 5065-5068; Johnson et al., Current Protocols in Nucleic Acid Chemistry 2004, 15(1), 13.1.1-13.1.31. However, there are challenges in employing these chemical methods, such as extensive reaction times and low yields. In many cases, phosphorylation of modified nucleosides at the required position is complex because other functional groups competing for the phosphate must be protected and then specifically removed after phosphorylation. Another popular chemical protocol described by Ludwig and Eckstein minimizes the generation of side products associated with use of POCl₃. Ludwig et al., Journal of Organic Chemistry 1989 54(3): 631-635. However, the method also requires the use of protecting groups and is sensitive to the quality of reagents. In many instances, absolutely dry reagents are required through co-evaporation with solvents such as anhydrous pyridine multiple times. Alternatively, nucleoside monophosphates (NMPs) have also been used as the starting materials, however, low yields are often obtained attributing to low reaction speed and material loss from purification of the complex crude mixture. Chambers et al. J. Am. Chem. Soc. 1957, 79(14): 3752-3755; Smith et al. J. Am. Chem. Soc. 1958, 80(5): 1141-1145. Unconverted reagents from these methods, such as NMPs, nucleotide diphosphates (NDP), pyrophosphate, and DCC, must be thoroughly removed to obtain the pure product.

Biosynthetic methods have been reported to produce canonical NTPs from nucleosides and NMPs. Kuwahara et al. Nucleic Acid Research 2006, 34(19): 5383-5394; Ladner et al. J. Org. Chem. 1985, 50(7): 1076-1079; Zhang et al. J. Microbiol. Biotechnol. 2015, 25(12): 2034-2042; Ding et al. Applied Biochemistry and Biotechnology 2020, 190, 1271-1288. However, there are few reports of modified nucleotide triphosphates synthesized by enzymatic means from NMPs. Accordingly, a need exists for an efficient, robust nucleoside phosphorylation process that is environmentally friendly, simple to execute, and yields high-purity products, particularly modified nucleotide triphosphates.

### SUMMARY

One aspect of the present application relates to a method of enzymatic synthesis of a nucleoside triphosphate having the structure of Formula (I):

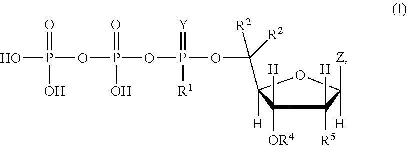

or a salt thereof,
the method comprising:
   (a) contacting a nucleoside monophosphate having the structure of Formula (II) or a salt thereof with a first phosphate donor in the presence of a nucleoside monophosphate (NMP) kinase to form a nucleoside diphosphate of Formula (III) or a salt thereof:

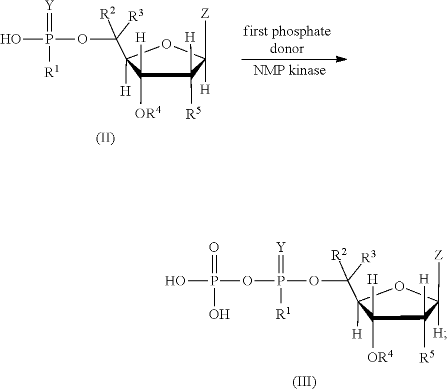

and
(b) reacting the nucleoside diphosphate of Formula (II) or the salt thereof with a second phosphate donor in the presence of a nucleoside diphosphate (NDP) kinase to form the nucleoside triphosphate of Formula (I) or the salt thereof:

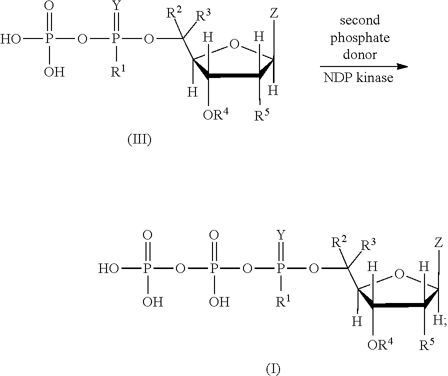

wherein:
R¹ is —OR^{a}, —SR^{a}, —NR^{b}R^{c}, or —BH₃;
each of R² and R³ is independently H, C₁-C₆ alkyl, C₁-C₆ hydroxyalkyl, —C(═O)H, —C(═O)OH, —N₃, —OR^{a}, —NR^{b}R^{c}, or

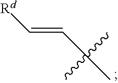

R⁴ is H, —CH₂N₃, —CHR^{a}N₃, —CH₂—CH═CH₂, —CH═CH₂, —CH₂—O—CH₂—CH═CH₂, or a 3′ hydroxy blocking group;
R⁵ is H, C₁-C₆ alkyl, vinyl, halo, —OR^{e} or a protected hydroxy group;
Y is O, S or Se;
each of R^{a}, R^{b} and R^{c} is independently H, or unsubstituted or substituted C₁-C₆ alkyl;
R^{d} is independently H, halo, C₁-C₆ alkyl, C₁-C₆ haloalkyl —CN, —C(═O)H, —C(═O)OH, —SO₂(C₁-C₆ alkyl), —N₃, —OH, or —NR^{b}R^{c};
R^{e} is H, unsubstituted or substituted C₁-C₆ alkyl, —CH₂N₃, —CHR^{a}N₃, —CH₂NR^{b}R^{c}, —CH₂—CH═CH₂, —CH═CH₂, or —CH₂—O—CH₂—CH═CH₂; and
Z is a natural nucleobase or a modified natural nucleobase;
provided that when Y is O, R¹ is —OH, R⁵ is OH or H, then at least one of R², R³ and R⁴ is not H.

Another aspect of the present application relates to stereospecific modified nucleoside triphosphate prepared by the enzymatic synthesis described herein.

### DETAILED DESCRIPTION

The methods disclosed herein relate to the preparation of nucleoside triphosphates from nucleoside monophosphates via enzymatic synthesis. In some embodiments, the nucleoside monophosphates may be modified at the nucleobase and/or at any position on the ribose or deoxyribose sugar moiety. In some embodiments, the nucleoside monophosphates and the respective nucleoside triphosphate products may include modification at the 2′, 3′ or 5′ position of the ribose or deoxyribose sugar moiety. The enzymatic synthesis described herein allows for the preparation of modified nucleotide triphosphates that would ordinarily be impractical to prepare using chemical methods.

### Definitions

The section headings used herein are for organizational purposes only and are not to be construed as limiting the subject matter described.

It is noted that, as used in this specification and the appended claims, the singular forms “a”, “an” and “the” include plural referents unless expressly and unequivocally limited to one referent. It will be apparent to those skilled in the art that various modifications and variations can be made to various embodiments described herein without departing from the spirit or scope of the present teachings. Thus, it is intended that the various embodiments described herein cover other modifications and variations within the scope of the appended claims and their equivalents.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
NTP Nucleotide triphosphate
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffN Fully functionalized nucleotide
RT Room temperature
SBS Sequencing by Synthesis
SM Starting material

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b”, inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃-C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. By way of example only, “C₁₋₆ alkyl” or “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as ““C₁₋₉ alkoxy” or “C₁-C₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. By way of example only, “C₂-C₆ alkenyl” or “C₂₋₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 6 carbon atoms. By way of example only, “C₂₋₆ alkynyl” or “C₂-C₆ alkenyl” indicates that there are two to six carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₆ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinolinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazolylalkyl, and imidazolylalkyl. In some cases, the alkylene group is a lower alkylene group (i.e., a C₁₋₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl”, “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, aziridinyl, azetidinyl, azepanyl, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “alkoxyalkyl” or “(alkoxy)alkyl” refers to an alkoxy group connected via an alkylene group, such as C₂-C₈ alkoxyalkyl, or (C₁-C₆ alkoxy) C₁-C₆ alkyl, for example, —(CH₂)₁₋₃—OCH₃.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “sulfino” group refers to a “—S(═O)OH” group.

A “sulfo” group refers to a “—S(═O)₂OH” or “—SO₃H” group.

A “sulfonate” group refers to a “—SO₃” group.

A “sulfate” group refers to “—SO₄” group.

A “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R^{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂-C₈ alkoxyalkyl” and the like.

As used herein, “—OAc” or “—O-acyl” refers to acetyloxy with the structure —O—C(═O)CH₃.

When a group is described as “optionally substituted” it may be either unsubstituted or substituted. Likewise, when a group is described as being “substituted”, the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl (C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, C₁-C₆ alkoxy (C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino (C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfonate, sulfate, sulfino, —OSO₂C₁-C₄alkyl, and oxo (═O). Wherever a group is described as “optionally substituted” that group can be substituted with the above substituents. In some embodiments, when an alkyl, alkenyl, alkynyl, aryl, heteroaryl, carbocyclyl or heterocyclyl group is substituted, each is independently substituted with one or more substituents selected from the group consisting of halo, —CN, —SO₃⁻, —OSO₃⁻, —SO₃H, —SR^{A}, —OR^{A}, —NR^{B}R^{C}, oxo, —CONR^{B}R^{C}, —SO₂NR^{B}R^{C}, —COOH, and —COOR^{B}, where R^{A}, R^{B} and R^{C} are each independently selected from H, alkyl, substituted alkyl, alkenyl, substituted alkenyl, alkynyl, substituted alkynyl, aryl, and substituted aryl.

As understood by one of ordinary skill in the art, a compound described herein may exist in ionized form, e.g., —CO₂⁻, —SO₃⁻ or —O—SO₃⁻. If a compound contains a positively or negatively charged substituent group, for example, —SO₃⁻, it may also contain a negatively or positively charged counterion such that the compound as a whole is neutral. For example, a compound may contain both —CO₂⁻ and a quaternary ammonium cation, or both —SO₃⁻ and a quaternary ammonium cation. In other aspects, the compound may exist in a salt form, where the counterion is provided by a conjugate acid or base. It is understood that when the compound described herein is substituted with —SO₃H, such substituent may exist in its anionic form —SO₃⁻ in aqueous solution.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two “adjacent” R groups are said to form a ring “together with the atom to which they are attached,” it is meant that the collective unit of the atoms, intervening bonds, and the two R groups are the recited ring. For example, when the following substructure is present:

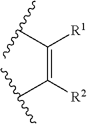

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

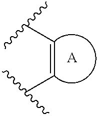

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

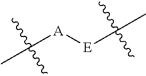

includes the substituent being oriented such that the A is attached at the leftmost attachment point of the molecule as well as the case in which A is attached at the rightmost attachment point of the molecule. In addition, if a group or substituent is depicted as

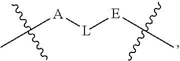

and L is defined an optionally present linker moiety; when L is not present (or absent), such group or substituent is equivalent to

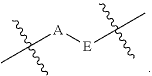

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e. a sugar lacking a hydroxy group that is present in ribose. The nitrogen containing heterocyclic base can be purine, deazapurine, or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleoside” is structurally similar to a nucleotide, but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art, and includes its tautomers. Similarly, the term “pyrimidine base” is used herein in its ordinary sense as understood by those skilled in the art, and includes its tautomers. A non-limiting list of optionally substituted purine-bases includes purine, adenine, guanine, deazapurine, 7-deaza adenine, 7-deaza guanine. hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, phosphoranilidate and phosphoramidate linkages. “Derivative”, “analog” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms (for example,

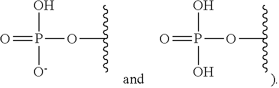 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Sometimes, “protecting group” and “blocking group” can be used interchangeably. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl (Bn); substituted benzyl; alkylcarbonyls (e.g., t-butoxycarbonyl (BOC), acetyl (i.e., —C(═O)CH₃ or Ac), or isobutyryl (iBu); arylalkylcarbonyls (e.g., benzyloxycarbonyl or benzoyl (i.e., —C(═O)Ph or Bz)); substituted methyl ether (e.g., methoxymethyl ether (MOM)); substituted ethyl ether (e,g., methoxyethyl ether (MOE); a substituted benzyl ether; tetrahydropyranyl ether; silyl ethers (e.g., trimethylsilyl (TMS), triethylsilyl, triisopropylsilyl, t-butyldimethylsilyl (TBDMS), tri-iso-propylsilyloxymethyl (TOM), or t-butyldiphenylsilyl); esters (e.g., benzoate ester); carbonates (e.g., methoxymethylcarbonate); sulfonates (e.g., tosylate or mesylate); acyclic ketal (e.g., dimethyl acetal); cyclic ketals (e.g., 1,3-dioxane or 1,3-dioxolanes); acyclic acetal; cyclic acetal; acyclic hemiacetal; cyclic hemiacetal; cyclic dithioketals (e.g., 1,3-dithiane or 1,3-dithiolane); and triarylmethyl groups (e.g., trityl; monomethoxytrityl (MMTr); 4,4′-dimethoxytrityl (DMTr); or 4,4′,4″-trimethoxytrityl (TMTr)).

Examples of hydroxy protecting groups include without limitation, acetyl, t-butyl, t-butoxymethyl, methoxymethyl, tetrahydropyranyl, 1-ethoxyethyl, 1-(2-chloroethoxy)ethyl, p-chlorophenyl, 2,4-dinitrophenyl, benzyl, 2,6-dichlorobenzyl, diphenylmethyl, p-nitrobenzyl, bis(2-acetoxyethoxy)methyl (ACE), 2-trimethylsilylethyl, trimethylsilyl, triethylsilyl, t-butyldimethylsilyl, t-butyldiphenylsilyl, triphenylsilyl, [(triisopropylsilyl)oxy]methyl (TOM), benzoylformate, chloroacetyl, trichloroacetyl, trifluoro-acetyl, pivaloyl, benzoyl, p-phenylbenzoyl, 9-fluorenylmethyl carbonate, mesylate, tosylate, triphenylmethyl (trityl), monomethoxytrityl, dimethoxytrityl (DMT), trimethoxytrityl, 1(2-fluorophenyl)-4-methoxypiperidin-4-yl (FPMP), 9-phenylxanthine-9-yl (Pixyl) and 9-(p-methoxyphenyl)xanthine-9-yl (MOX). Wherein more commonly used hydroxyl protecting groups include without limitation, benzyl, 2,6-dichlorobenzyl, t-butyldimethylsilyl, t-butyldiphenylsilyl, benzoyl, mesylate, tosylate, dimethoxytrityl (DMT), 9-phenylxanthine-9-yl (Pixyl) and 9-(p-methoxyphenyl) xanthine-9-yl (MOX).

### Enzymatic Synthesis of Modified Nucleoside Triphosphates

Some embodiments of the present disclosure relate to a method of enzymatic synthesis of a nucleoside triphosphate having the structure of Formula (I):

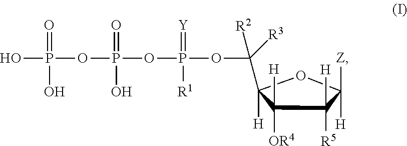

or a salt thereof,
the method comprising:
   (a) contacting a nucleoside monophosphate having the structure of Formula (II) or a salt thereof with a first phosphate donor in the presence of a nucleoside monophosphate (NMP) kinase to form a nucleoside diphosphate of Formula (III) or a salt thereof, and
   (b) reacting the nucleoside diphosphate of Formula (II) or the salt thereof with a second phosphate donor in the presence of a nucleoside diphosphate (NDP) kinase to form the nucleoside triphosphate of Formula (I) or the salt thereof;

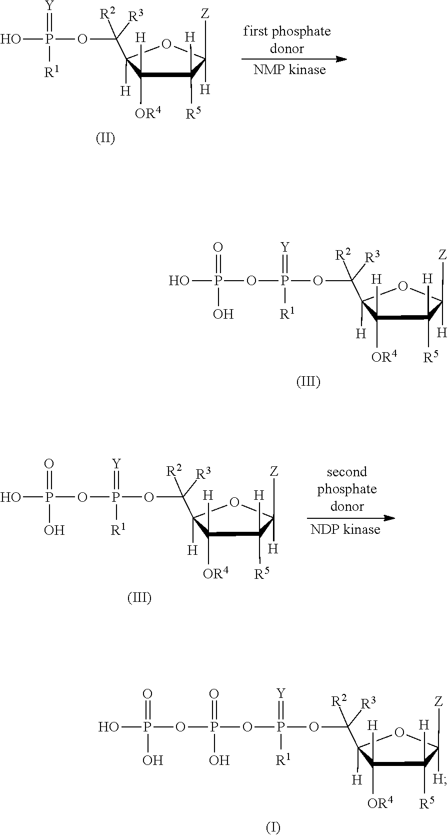

wherein:
R¹ is —OR^{a}, —SR^{a}, —NR^{b}R^{c}, or —BH₃;
each of R² and R³ is independently H, C₁-C₆ alkyl, C₁-C₆ hydroxyalkyl, —C(═O)H, —C(═O)OH, —N₃, —OR^{a}, —NR^{b}R^{c}, or

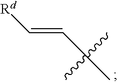

R⁴ is H, —CH₂N₃, —CHR^{a}N₃, —CH₂—CH═CH₂, —CH═CH₂, —CH₂—O—CH₂—CH═CH₂, or a 3′ hydroxy blocking group;
R⁵ is H, C₁-C₆ alkyl, vinyl, halo, —OR^{e} or a protected hydroxy group;
Y is O, S or Se;
each of R^{a}, R^{b} and R^{c} is independently H, or unsubstituted or substituted C₁-C₆ alkyl;
R^{d} is independently H, halo, C₁-C₆ alkyl, C₁-C₆ haloalkyl-CN, —C(═O)H, —C(═O)OH, —SO₂(C₁-C₆ alkyl), —N₃, —OH, or —NR^{b}R^{c};
R^{e} is H, unsubstituted or substituted C₁-C₆ alkyl, —CH₂N₃, —CHR^{a}N₃, —CH₂NR^{b}R^{c}, —CH₂—CH═CH₂, —CH═CH₂, or —CH₂—O—CH₂—CH═CH₂; and
Z is a natural nucleobase or a modified natural nucleobase;
provided that when Y is O, R¹ is —OH, R⁵ is OH or H, then at least one of R², R³ and R⁴ is not H.

Some further embodiments of the present disclosure relate to a method of enzymatic synthesis of a nucleoside diphosphate having the structure of Formula (III) as described herein, the method includes reacting the nucleoside monophosphate having the structure of Formula (II) as described herein or a salt thereof with a first phosphate donor in the presence of a nucleoside monophosphate (NMP) kinase to form the nucleoside diphosphate of Formula (III) or a salt thereof.

Some further embodiments of the present disclosure relate to a method of enzymatic synthesis of a nucleoside triphosphate having the structure of Formula (I) as described herein, the method includes reacting the nucleoside diphosphate of Formula (II) as described herein or the salt thereof with a second phosphate donor in the presence of a nucleoside diphosphate (NDP) kinase to form the nucleoside triphosphate of Formula (I) as described herein or the salt thereof.

In some embodiments of the methods described herein, the first phosphate donor comprises or is adenosine triphosphate (ATP) or deoxyadenosine triphosphate (dATP). In one embodiment, the first phosphate donor is ATP. In some embodiments, the NMP kinase comprises bovine adenylate kinase (AKB), porcine adenylate kinase (AKP), Escheria E. Coli adenylate kinase (AKE), yeast guanylate kinase (GKY), or T4 deoxynucleotide monophosphate (DNMP) kinase, thymidylate kinase, cytidylate kinase, uridylate kinase, or a combination thereof. In one embodiment, the NMP kinase is T4 DNMP kinase. In some embodiments, the NMP kinase catalyzed phosphorylation of the nucleoside monophosphate is also in the presence of a divalent cation. In some such embodiments, the divalent cation comprises or is Mg²⁺. NMP kinases require divalent cations, such as Mg²⁺, to perform the phosphoryl transfer. While it is widely known in polymerase mediated incorporation of xeno nucleic acids that other metal salts can improve catalytic activity and reduce specificity, there is less precedence for NMP kinases. Larger cationic metals may be used to expand the catalytic site of the NMP binding domain on the kinase to tolerate larger modifications present on the NMPs. Larger cationic metals include but not limited to Mn²⁺, Ca²⁺, Zn²⁺ and Cu²⁺. In some embodiments, the molar ratio of the nucleoside monophosphate of Formula (II) to the NMP kinase (e.g., T4-dNMP kinase) is about 5000:1, 4500:1, 4000:1, 3500:1, 3000:1, 2900:1, 2800:1, 2700:1, 2600:1, 2500:1, 2400:1, 2300:1, 2200:1, 2100:1, 2000:1, 1900:1, 1800:1, 1700:1, 1600:1, 1500:1, 1400:1, 1300:1, 1200:1, 1100:1, 1000:1, 900:1, 800:1, 700:1, 600:1, 500:1, 400:1, 300:1, 200:1, 100:1, 90:1, 80:1, 70:1, 60:1, 50:1, 40:1, 30:1, 20:1, 10:1, 9:1, 8:1, 7:1, 6:1, 5:1, 4:1, 3:1, 2:1 or 1:1, or a range defined by any two of the preceding values. In some further embodiments, the molar ratio of the nucleoside monophosphate of Formula (II) to the NMP kinase (e.g., T4-dNMP kinase) is from about 3000:1 to about 2400:1, from about 2800:1 to about 2600:1, or about 2700:1.

In some embodiments of the methods described herein, the NDP kinase comprises or is creatine kinase, pyruvate kinase, succinyl-CoA synthetase, or acetate kinase. In some embodiments, the second phosphate donor comprises ATP, creatine phosphate or acetyl phosphate, or a combination thereof. In some further embodiments, the NDP kinase is creatine kinase, and the second phosphate donor is creatine phosphate. In some other embodiments, the NDP kinase is acetate kinase, and the second phosphate donor is acetate phosphate. In some embodiments, step (a) and step (b) is a one-pot reaction in a single reaction vessel. In some embodiments, the one-pot reaction is conducted at a temperature from room temperature to about 65° C., or heated to a temperature of about 30° C. to about 40° C., or about 37° C. In some further embodiments, the reaction vessel is heated for from about 1 hour to about 24 hours, from about 4 hours to about 20 hours, or about 12 hours. In some embodiments, the molar ratio of the nucleoside diphosphate of Formula (III) to the NDP kinase (e.g., creatine kinase) is about 5000:1, 4500:1, 4000:1, 3500:1, 3000:1, 2900:1, 2800:1, 2700:1, 2600:1, 2500:1, 2400:1, 2300:1, 2200:1, 2100:1, 2000:1, 1900:1, 1800:1, 1700:1, 1600:1, 1500:1, 1400:1, 1300:1, 1200:1, 1100:1, 1000:1, 900:1, 800:1, 700:1, 600:1, 500:1, 400:1, 300:1, 200:1, 150:1, 100:1, 90:1, 85:1, 80:1, 75:1, 70:1, 65:1, 60:1, 55:1, 50:1, 45:1, 40:1, 35:1, 30:1, 25:1, 20:1, 15:1, 10:1, 9:1, 8:1, 7:1, 6:1, 5:1, 4:1, 3:1, 2:1 or 1:1, or a range defined by any two of the preceding values. In some further embodiments, the molar ratio of the nucleoside diphosphate of Formula (III) to the NDP kinase (e.g., creatine kinase) is from about 50:1 to about 5:1, or from about 25:1 to about 10:1, or about 20:1 to about 15:1.

The use of NMP kinases (NMP Ks) with less substrate specificity are important to catalytic generation of NDPs from stable NMPs. For example, bacteriophage T4 deoxynucleotide monophosphate (DNMP) kinase (T4 kinase) is unique in its ability to recognize three structurally dissimilar mononucleotides: dGMP, dTMP, and 5′-hydroxymethyl-dCMP. In order to obtain structurally diverse modified NTPs, variants of these kinases and mutations on important sites can be used, especially on the NMP binding domain. These enzymes, when paired with NDP kinases (NDP Ks) for phosphorylation to the triphosphate form, can generate triphosphates at about 5 μmol to about 5 mmol/hr in a solution of about 1 mL, achieving a conversion rate of about or at least about 25%, 30%, 35%, 40%, 45%, 50%, 55%, 60%, 65%, 70%, 75%, 80%, 85%, 90%, 92%, 95%, 98%, 99%, or a range defined by any two of the preceding values. Some advantages of this method over a chemical synthesis method include the simplicity of combining reagents in a single pot with minimal preparation time and cost, mild reaction conditions, the ability to generate NTPs at a high throughput, and the ability to access modifications sensitive to traditional chemical synthesis methods. Enzymatic synthesis can be used to generate stereospecific NTPs. This method can also eliminate the need to thoroughly remove unconverted reagents or byproducts in order to isolate a pure product.

The enzymatic synthesis method described herein can be adapted to sequencing systems where modified NTPs are generated in situ or as part of an environmentally benign production process from stable NMPs. Use of purified enzymes can minimize side reactions and simplify reaction workups. Furthermore, phosphate donor recycling systems can increase yields in these reactions by shifting the reaction equilibrium toward the desired product NTPs.

In some embodiments of the methods described herein, the starting materials for making the NTPs described herein are modified nucleoside monophosphate. Possible positions for modification include the 5′, 3′, or 2′ on the ribose moiety as well as nucleobase modification, modification on the monophosphate, or combinations thereof. In some embodiments of the compound of Formula (I), (II) or (III), Y is O. In some other embodiments, Y is S. In some embodiments, R¹ is —OH. In other embodiments, R¹ is —O⁻ so the mono-, di-, or tri-phosphate is in a salt form (e.g., a sodium or potassium salt). In some other embodiments, R¹ is BH₃. In still other embodiments, R¹ is —NHR^{c}. In some embodiments, each of R² and R³ is H. In other embodiments, R² is H and R³ is C₁-C₆ hydroxyalkyl such as —CH₂OH. In other embodiments, R² is H and R³ is a substituted vinyl having the structure:

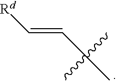

In some embodiments, R⁴ is not H. For example, R⁴ is a 3′hydroxy blocking group such as —CH₂N₃, —CHR^{a}N₃, —CH₂—CH═CH₂, —CH═CH₂, or —CH₂—O—CH₂—CH═CH₂. In one embodiment, R⁴ is —CH═CH₂. In some embodiments, R⁵ is H. In other embodiments, R⁵ is C₁-C₆ alkyl (e.g., methyl or ethyl), halo(e.g., F), —OR^{e} (e.g., —OCH₃), or a protected hydroxy group. In some embodiments, Z is

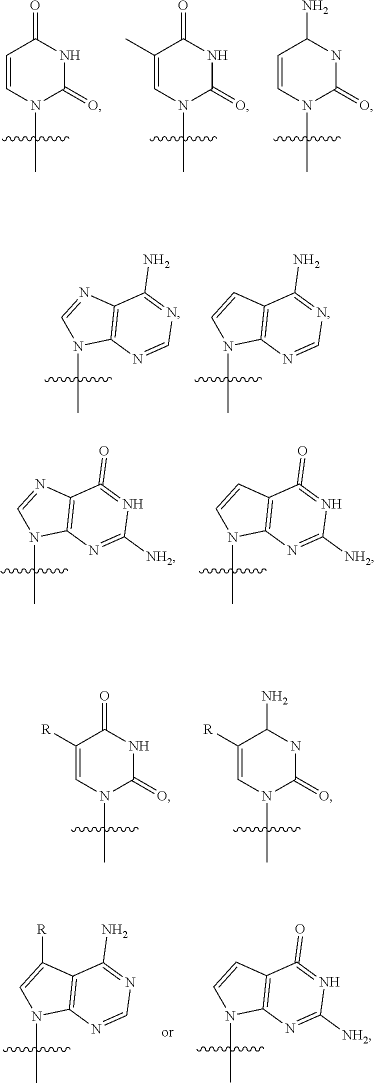

wherein R comprises a functional moiety or a detectable label attached to the nucleobase optionally through a linker such as a cleavable linker.

### Cleavable Linkers

Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO2004/018493 (herein incorporated by reference) such as those that include moieties of the formulae:

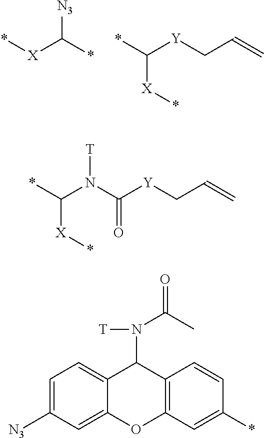

(wherein X is selected from the group comprising O, S, NH and NQ wherein Q is a C₁₋₁₀ substituted or unsubstituted alkyl group, Y is selected from the group comprising O, S, NH and N(allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels such as, for example, the dye compounds described herein.

Additional examples of linkers include those disclosed in U.S. Publication No. 2016/0040225 (herein incorporated by reference), such as those include moieties of the formulae:

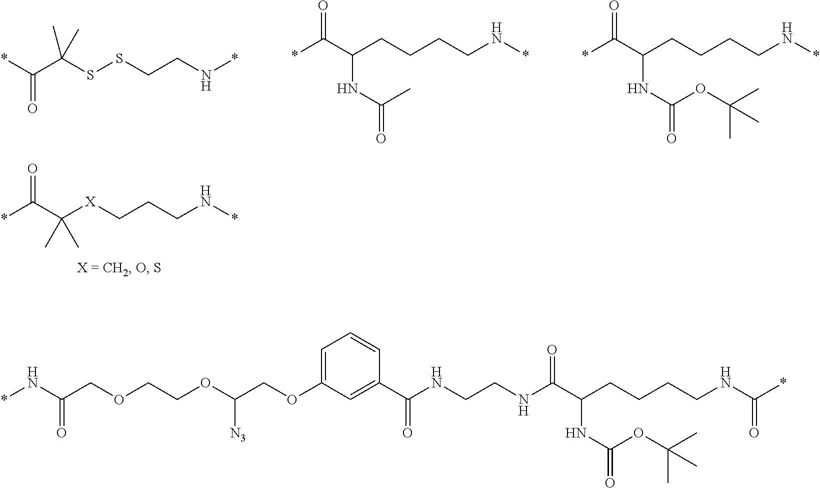

(wherein * indicates where the moiety is connected to the remainder of the nucleotide or nucleoside). The linker moieties illustrated herein may comprise the whole or partial linker structure between the nucleotides/nucleosides and the labels.

Additional examples of linkers are disclosed in U.S. Publication No. 2020/0216891 A1, which is incorporated by reference in its entirety:

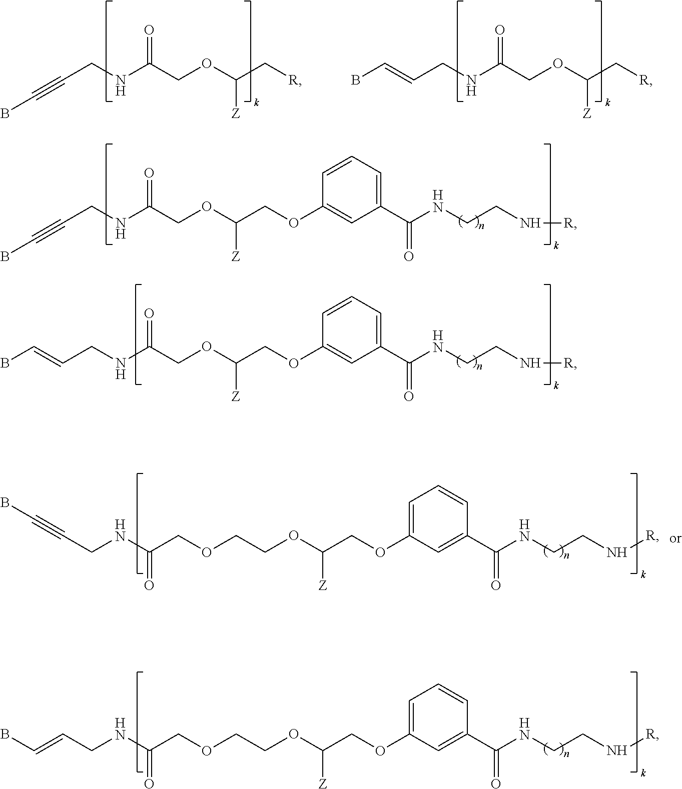

wherein B is a nucleobase (Z) described herein; n is 1, 2, 3, 4, 5; k is 1; Z is —N₃ (azido), —O—C₁-C₆ alkyl, —O—C₂-C₆ alkenyl, or —O—C₂-C₆ alkynyl; and R comprises the functional moiety or detectable label described herein, which may contain additional linker and/or spacer structure. In one embodiment, the cleavable linker comprises

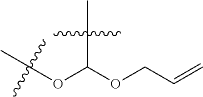 [SMILES: *OC(*)OCC=C]

(“AOL” linker moiety) where Z is —O-allyl. For the purpose of the present disclosure, the nucleotide may contain multiple cleavable linkers repeating units (e.g., k is 1, 2, 3, 4, 5, 6, 7, 8, 9 or 10).

In some further embodiments, Z comprises

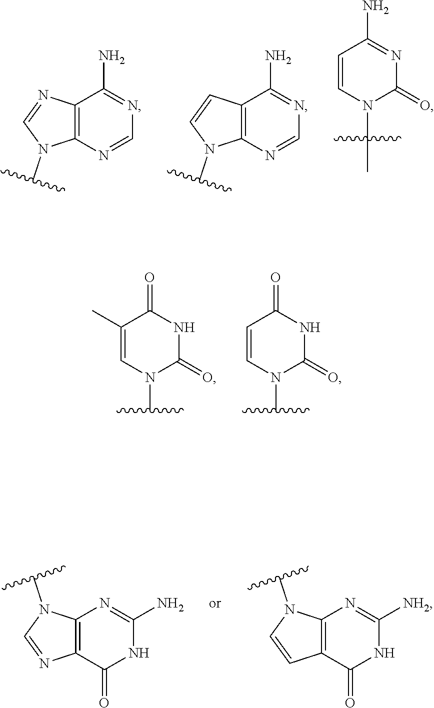

or optionally substituted derivatives and analogs thereof. In some further embodiments, the nucleobase Z comprises the structure

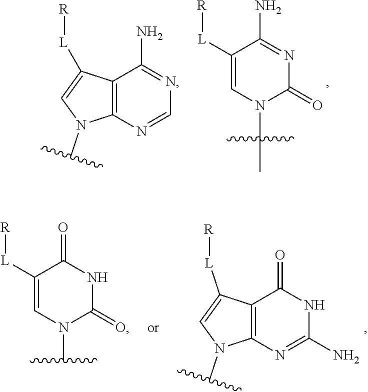

wherein L is the linker or cleavable linker described herein.

In any embodiments of the methods described herein, the modified nucleoside monophosphate of Formula (I) is:

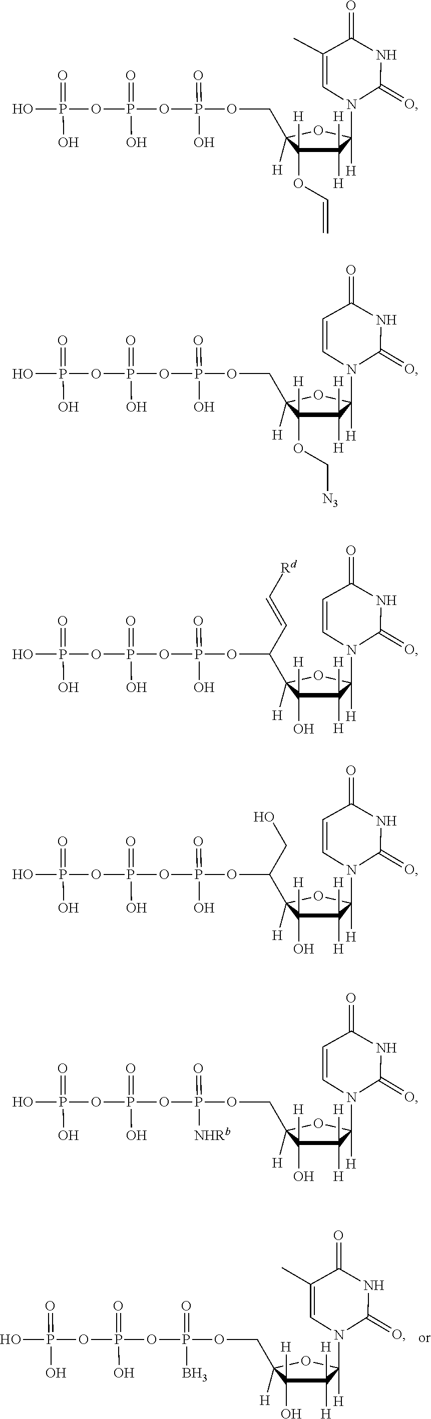

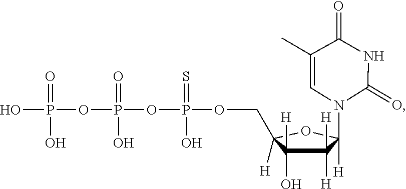

or a salt thereof

In any embodiments of the methods described herein, the NMP kinase catalyzed phosphorylation of the modified nucleoside monophosphate results in at least 95%, 98%, or 99% enantiomerically pure nucleoside diphosphate. In further embodiments, the NDP kinase catalyzed phosphorylation of the modified nucleoside diphosphate results in at least 95%, 98%, or 99% enantiomerically pure nucleoside triphosphate.

### Examples

Some aspects of the embodiments discussed above are disclosed in further detail in the following examples, which are not in any way intended to limit the scope of the present disclosure. Those in the art will appreciate that many other embodiments also fall within the scope of the compositions, kits and methods of the present application, as is described herein above and in the claims.

Example 1. Synthesis of 3′Vinyl Blocked dTMP

A 3′-vinyl protected T nucleoside (Compound 3) was synthesized by two different synthetic routes illustrated in Schemes 1 and Scheme 2 respectively.

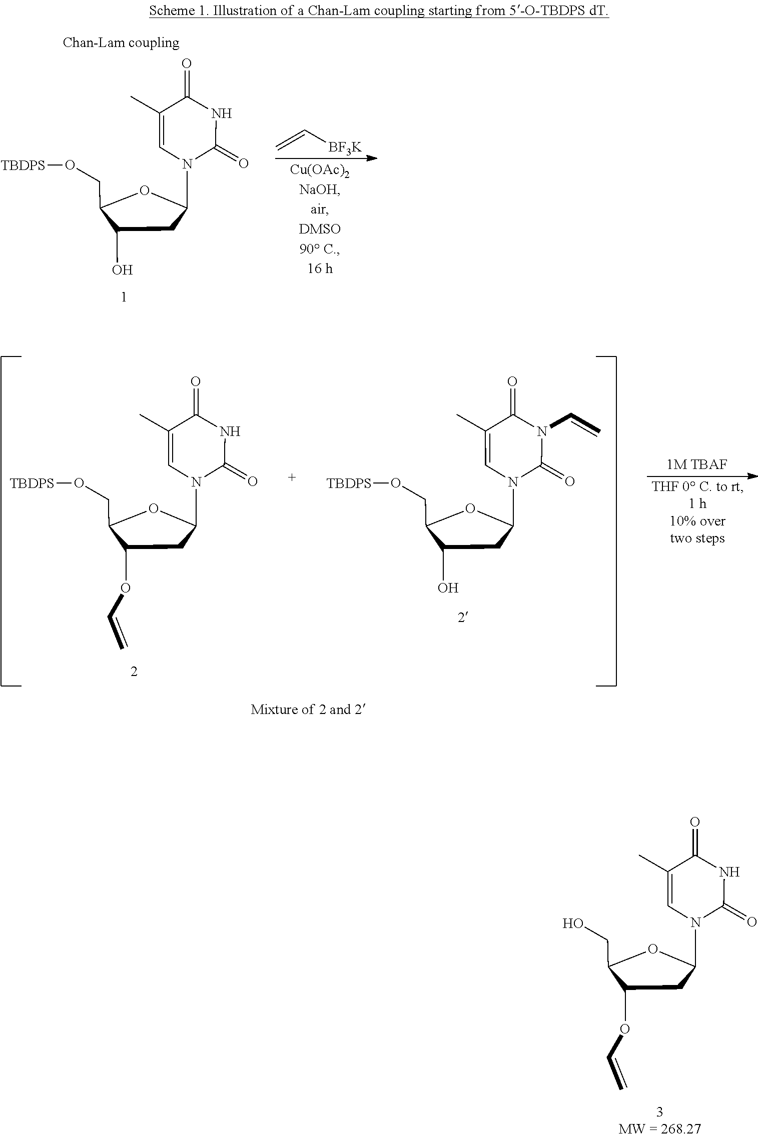

To a solution of 5′-O-TBDPS-dT nucleoside 1 (0.5 mmol, 480.64 g/mol) in DMSO containing molecular sieves (10.0 mL), under air atmosphere and at room temperature were added potassium vinyl trifluoroborate (1 mmol, 133.95 g/mol), copper acetate (10 mol %, 123.60 g/mol) and powdered NaOH (1.0 mmol, 40 g/mol). The reaction mixture was heated gently to 90° C. for 16 h at which point TLC showed the complete conversion of alcohol 1 to the vinylated products 2 and 2′. After cooling to room temperature, ice cold water was added dropwise to the reaction mixture, then filtered and diluted with EtOAc. The organic layer was washed twice with water and brine before being dried over anhydrous sodium sulphate. The drying agent was removed by filtration, and the solvent was evaporated under vacuum to give a crude colorless oil which was directly dissolved in THF, cooled to 0° C. and treated with TBAF (1 mmol, 1M). The reaction mixture was then warmed to room temperature and stirred for additional 1 h, quenched with ice cold water and diluted with EtOAC. The organic layer was washed twice with water and brine before being dried over anhydrous sodium sulphate. The drying agent was removed by filtration and the solvent was evaporated under vacuum to give a crude colorless oil purified by silica gel chromatography (hexane/EtOAc=2:3) to yield 3′-vinyl compound 3 as a pale-yellow oil (10% over two steps). The purified fractions were characterized by ¹H NMR and MS to confirm the structure 3. ESI-MS (− ve mode) m/z 267.1 [M−H]⁻.

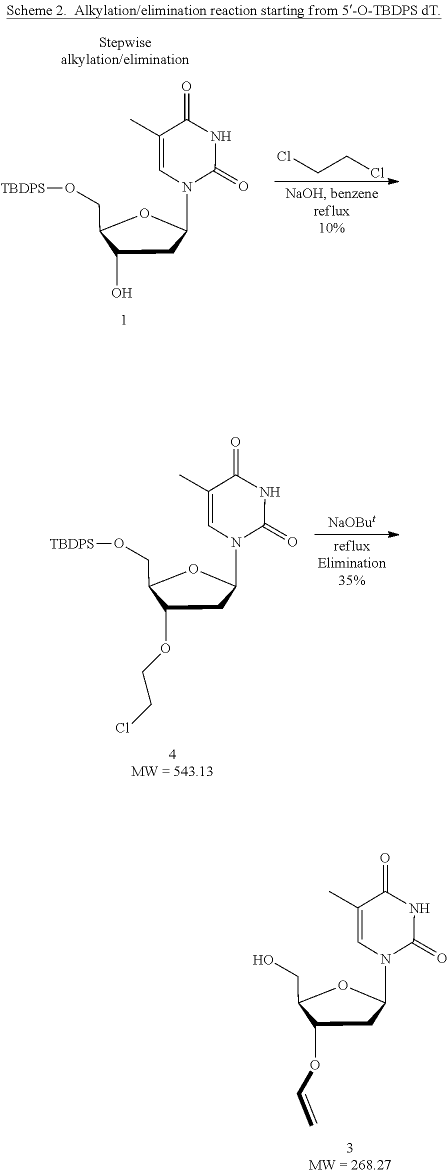

To a solution of 5′-O-TBDPS-dT nucleoside 1 (5.41 mmol, 480.64 g/mol) in benzene (20 mL) and 1,2-dichloroethane (10.0 mL) was added powdered NaOH (10.82 mmol, 40 g/mol) under argon atmosphere at room temperature. The reaction mixture was gently refluxed to 90° C. for 16 h to 76 h, at which point TLC showed ˜15% conversion of compound 1 to the 3′-alkylated product 4. After cooling to room temperature, ice cold water was added dropwise to the reaction mixture, then filtered and diluted with EtOAc. The organic layer was washed twice with water and brine before being dried over anhydrous sodium sulphate. The drying agent was removed by filtration and the solvent was evaporated under vacuum to give a crude colorless oil, purified by silica gel chromatography (hexane/EtOAc=1:4) to yield alkylated compound 4 as pale-yellow oil (10%). The purified fractions were characterized by ¹H NMR and MS to confirm the structure 4. ESI-MS (− ve mode) m/z 542.2 [M−H]⁻.

To ether 4 (0.184 mmol, 542.2 g/mol) dissolved in THF (3.0 mL) was added sodium tert-butoxide (1 mmol, 96.10 g/mol). The reaction mixture was gently refluxed for 3 h at which point TLC showed the complete conversion to the vinylated compound 3. After cooling to room temperature, ice cold water was added dropwise to the reaction mixture, then filtered and diluted with EtOAc. The organic layer was washed twice with water and brine before being dried over anhydrous sodium sulphate. The drying agent was removed by filtration and the solvent was evaporated under vacuum to give a crude colorless oil purified by silica gel chromatography (hexane/EtOAc=2:3) to yield 3′-vinyl compound 3 as a pale-yellow oil (35%). The purified fractions were characterized by ¹H NMR and MS to confirm the structure of compound 3.

Compound 3 was subsequently used as a starting point to synthesize 3′-vinyl deoxythymidine monophosphate (dTMP), which was illustrated in Scheme 3.

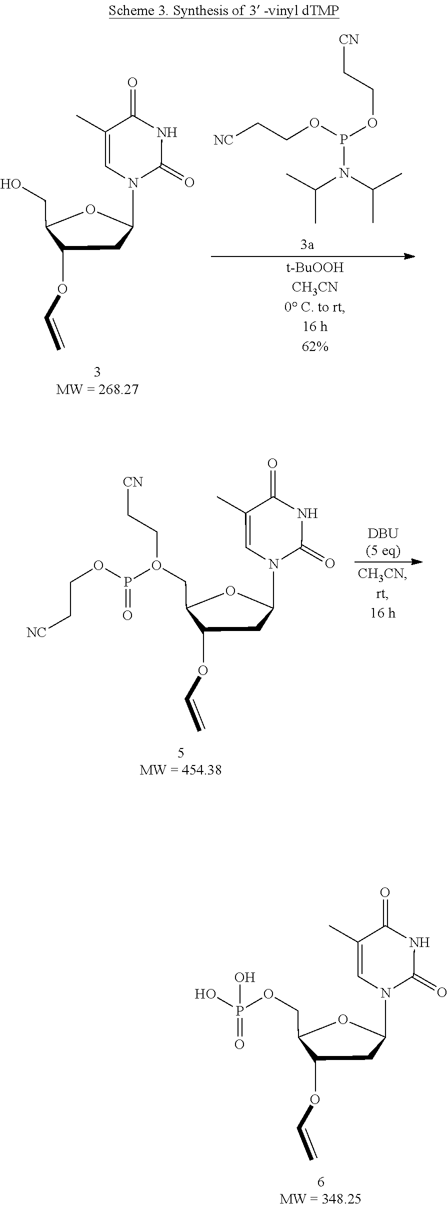

Alcohol 3 (75 μmol, 268 g/mol) and 5-(ethylthio)-1H-tetrazole (110 μmol, 130.17 g/mol) were co-evaporated with dry acetonitrile (3×1 mL) before being dissolved in CH₂Cl₂/CH₃CN (1:1, 4 mL). Under an atmosphere of dry argon, a freshly prepared reaction mixture of phosphoramidite 3a (110 μmol, 271.3 g/mol.) in acetonitrile (2 mL) was added to the above reaction mixture and stirred overnight at room temperature at which point TLC showed the complete conversion of alcohol 3 to phosphorylated compound 5. Upon cooling to 0° C., t-BuOOH (110 μmol, 6 M) was added and the mixture was stirred for 5 min at 0° C. until TLC confirmed complete oxidation. The solvent was removed under vacuum to give a crude colorless oil which was purified by silica gel chromatography (hexane/EtOAc=1:4) to yield phosphorylated product 5 as an off-white foam (62%).

To phosphorylated compound 5 (110 μmol, 454.38 g/mol) dissolved in CH₃CN (200 μL) under an atmosphere of dry argon was added DBU (550 μmol, 0.1 M in acetonitrile) and the reaction mixture was stirred overnight at room temperature at which point TLC showed the complete conversion to monophosphate 6. The solvent was removed under the vacuum to give a crude colorless oil which was purified by RP-HPLC. The purified fractions were characterized by MS and lyophilized to obtain monophosphate 6.

### Example 2. One-Pot Synthesis of 3′-Vinyl-Deoxythymidine Triphosphate

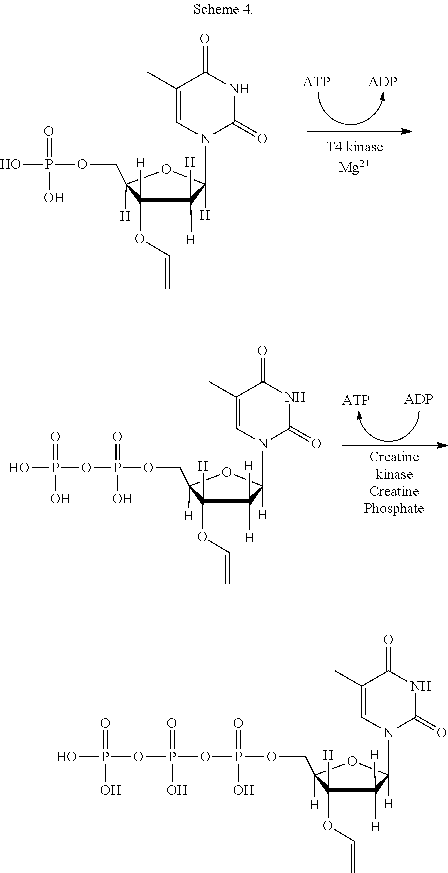 [SMILES: C=CO[C@@H]1C[C@H](n2cc(C)c(=O)[nH]c2=O)O[C@@H]1COP(=O)(O)O ; C=CO[C@@H]1C[C@H](n2cc(C)c(=O)[nH]c2=O)O[C@@H]1COP(=O)(O)OP(=O)(O)O ; C=CO[C@@H]1C[C@H](n2cc(C)c(=O)[nH]c2=O)O[C@@H]1COP(=O)(O)OP(=O)(O)OP(=O)(O)O]

To a 200 μL PCR reaction tube, the following components were added in the stated order to make up a 100 μL one-pot synthesis reaction: 2 μL of 100 mM 3′-vinyl-deoxythymidine monophosphate (3′vinyl-dTMP), 10 μL 10XT4 kinase buffer (10X: 70 mM Tris pH7.5, 10 mM MgCl₂), 8 μL of 10 mM adenosine triphosphate (ATP), 50 μL of 20 mg/mL of creatine kinase, 7.3 μL of 110 mM of phosphocreatine di(tris)salt solution, 21.7 μL of water, 1 μL of T4 dNMP kinase (200 U/μL), and 50 μL of Creatine Kinase (20 mg/ml). The tube was heated overnight at 37° C. An aliquot from the tube was taken and analyzed via LC-MS, showing conversion of the 3′vinyl-dTMP to the corresponding diphosphate and triphosphate form. While the 3′-vinyl group is chemically sensitive, the enzymatic synthesis left the 3′-vinyl group intact during the phosphorylation reaction. In contrast, such 3′ blocking group would have been removed if chemical methods using POCl₃ or salicyl phosphorochloridite had been used.

While the present application has been described with reference to the specific embodiments thereof, it should be understood by those skilled in the art that various changes may be made and equivalents may be substituted without departing from the true spirit and scope of the present application. In addition, many modifications may be made to adapt a particular situation, material, composition of matter, process, process step or steps, to the objective, spirit and scope of the present application. All such modifications are intended to be within the scope of the claims appended hereto.

1. A method of enzymatic synthesis of a nucleoside triphosphate having the structure of Formula (I): 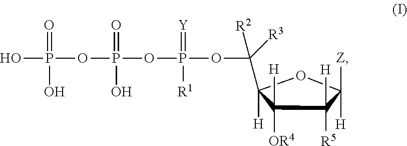
or a salt thereof,
the method comprising:
(a) contacting a nucleoside monophosphate having the structure of Formula (II) or a salt thereof with a first phosphate donor in the presence of a nucleoside monophosphate (NMP) kinase to form a nucleoside diphosphate of Formula (III) or a salt thereof: 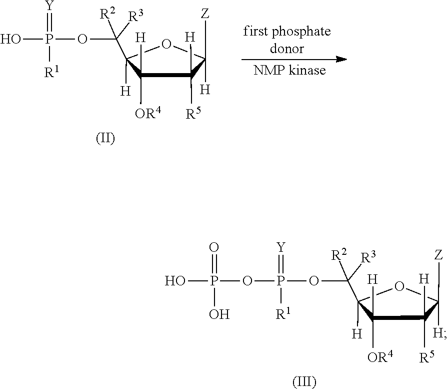
and
(b) reacting the nucleoside diphosphate of Formula (II) or the salt thereof with a second phosphate donor in the presence of a nucleoside diphosphate (NDP) kinase to form the nucleoside triphosphate of Formula (I) or the salt thereof: 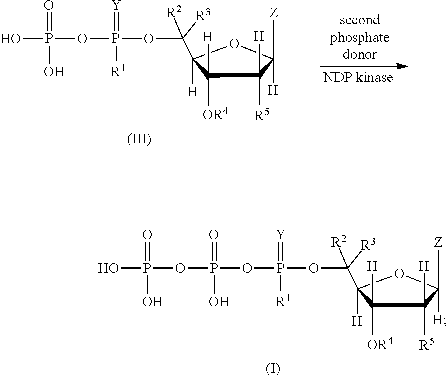
wherein:
R¹ is —OR^{a}, —SR^{a}, —NR^{b}R^{c}, or —BH₃;
each of R² and R³ is independently H, C₁-C₆ alkyl, C₁-C₆ hydroxyalkyl, —C(═O)H, —C(═O)OH, —N₃, —OR^{a}, —NR^{b}R^{c}, or 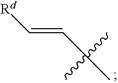
R⁴ is H, —CH₂N₃, —CHR^{a}N₃, —CH₂—CH═CH₂, —CH═CH₂, —CH₂—O—CH₂—CH═CH₂, or a 3′ hydroxy blocking group;
R⁵ is H, C₁-C₆ alkyl, vinyl, halo, —OR^{e} or a protected hydroxy group;
Y is O, S or Se;
each of R^{a}, R^{b} and R^{c} is independently H, or unsubstituted or substituted C₁-C₆ alkyl;
R^{d} is independently H, halo, C₁-C₆ alkyl, C₁-C₆ haloalkyl —CN, —C(═O)H, —C(═O)OH, —SO₂ (C₁-C₆ alkyl), —N₃, —OH, or —NR^{b}R^{c};
R^{e} is H, unsubstituted or substituted C₁-C₆ alkyl, —CH₂N₃, —CHR^{a}N₃, —CH₂NR^{b}R^{c}, —CH₂—CH═CH₂, —CH═CH₂, or —CH₂—O—CH₂—CH═CH₂; and
Z is a natural nucleobase or a modified natural nucleobase;
provided that when Y is O, R¹ is —OH, R⁵ is OH or H, then at least one of R², R³ and R⁴ is not H.

2. The method of claim 1, wherein the first phosphate donor is adenosine triphosphate (ATP) or deoxyadenosine triphosphate (dATP).

3. The method of claim 2, wherein the NMP kinase comprises bovine adenylate kinase (AKB), porcine adenylate kinase (AKP), Escheria E. Coli adenylate kinase (AKE), yeast guanylate kinase (GKY), T4 deoxynucleotide monophosphate (DNMP) kinase, thymidylate kinase, cytidylate kinase, or uridylate kinase, or a combination thereof.

4. The method of claim 3, wherein the NMP kinase is T4 DNMP kinase.

5. The method of claim 3, wherein the molar ratio of the NMP kinase to the nucleoside monophosphate is from about 3000:1 to about 2000:1, or from about 2800:1 to about 2400:1.

6. The method of claim 1, wherein the NMP kinase catalyzed phosphorylation of the nucleoside monophosphate in step (a) is also in the presence of a divalent cation.

7. The method of claim 6, wherein the divalent cation comprises Mg²⁺.

8. The method of claim 1, wherein the NDP kinase comprises creatine kinase, acetate kinase, pyruvate kinase, or succinyl-CoA synthetase, or a combination thereof.

9. The method of claim 8, wherein the second phosphate donor comprises ATP, creatine phosphate or acetyl phosphate, or a combination thereof.

10. The method of claim 8, wherein the NDP kinase is creatine kinase, and the second phosphate donor is creatine phosphate.

11. The method of claim 10, wherein the molar ratio of the creatine kinase to the nucleoside diphosphate is from about 20:1 to about 10:1 or from about 18:1 to about 14:1.

12. The method of claim 1, wherein step (a) and step (b) is a one-pot reaction in a single reaction vessel, heated to a temperature of about 30° C. to about 40° C.

13. (canceled)

14. (canceled)

15. The method of claim 12, wherein the one-pot reaction is heated for about 1 hour to about 24 hours.

16. (canceled)

17. The method of claim 1, wherein Y is O.

18. The method of claim 1, wherein R¹ is —OH.

19. The method of claim 1, wherein each of R² and R³ is H.

20. The method of claim 1, wherein R⁴ is a 3′ hydroxy blocking group.

21. The method of claim 20, wherein R⁴ is —CH═CH₂.

22. The method of claim 1, wherein R⁵ is H, C₁-C₆ alkyl, or halo.

23. The method of claim 1, wherein Z is 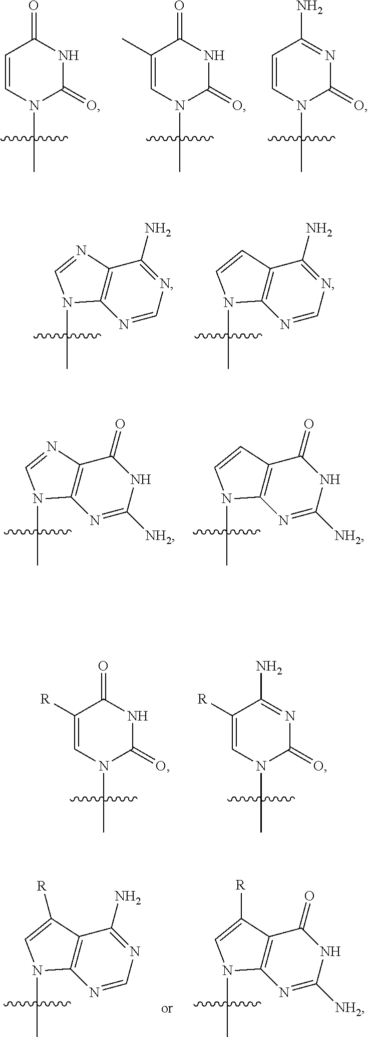
wherein R comprises a functional moiety or a detectable label attached to the nucleobase optionally through a linker.

24. The method of claim 1, wherein the nucleoside monophosphate of Formula (I) is: 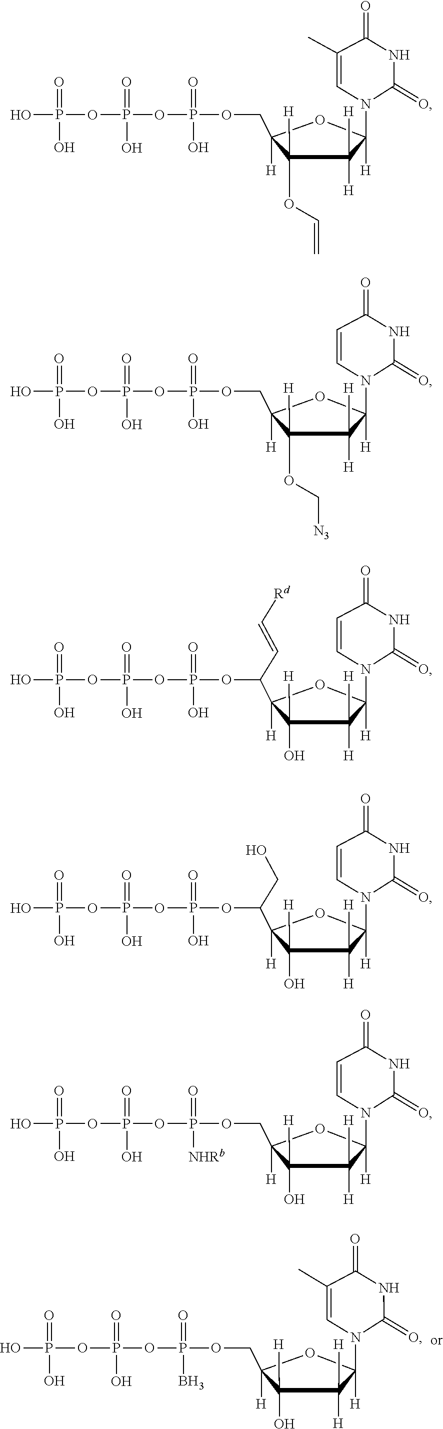 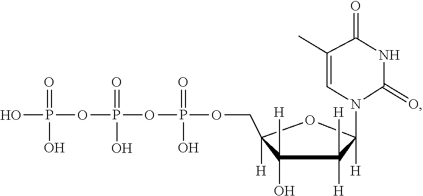 [SMILES: Cc1cn([C@H]2C[C@@H](O)[C@@H](COP(=O)(O)OP(=O)(O)OP(=O)(O)O)O2)c(=O)[nH]c1=O]
or a salt thereof.
